SEQUENCE LISTING (46 sequences)

SEQ ID NO: 1 - DNA, 1596 bp, Gluconobacter oxydans
atgacgagcggttttgattacatcgttgtcggtggcggttcggctggctgtgttctcgcagcccgcctttccgaaaatccttccgtccgtgtctgcctcatcgaggcgggccggcgggacacgcatcccctgatccatatgccggtcggtttcgcgaagatgaccacggggccgcatacctgggatcttctgacggagccgcagaaacatgcgaacaaccgccagatcccctatgtgcagggccggattctgggcggcggatcgtccatcaacgcggaagtcttcacgcggggacacccttccgatttcgaccgctgggcggcggaaggtgcggatggctggagcttccgggatgtccagaagtacttcatccgttccgaaggcaatgccgtgttttcgggcacctggcatggcacgaacgggccgctcggggtgtccaacctcgcagatccgaacccgaccagccgtgccttcgtgcagagctgtcaggaaatggggctgccctacaaccctgacttcaatggcgcatcgcaggaaggggctggcatctaccagatgaccatccggaacaaccggcgctgctcgacggctgtggggtatctgcgtccggccctggggcggaagaacctgacggttgtgacgcgggcgctggtcctgaagatcgtcttcaacgggacgcgggcgacgggcgtgcagtatatcgccaacggcaccctgaataccgccgaagcgagccaggaaatcgttgtgacggccggagcgatcggaacgccgaagctgatgatgctgtcgggcgtcgggcctgccgcgcatcttcgcgaaaatggtatcccggtcgtgcaggatctgccgggcgtgggcgagaaccttcaggaccatttcggtgtggatatcgtagccgagctcaagacggatgagagcttcgacaagtaccggaaactgcactggatgctgtgggcaggtcttgaatacaccatgttcagatccggccccgtcgcgtccaacgtggttgagggcggcgcgttctggtactcggacccgtcatcgggtgttcctgatctccagttccattttcttgcgggggcaggggctgaggcaggggtgacgtccgttcccaagggcgcgtcggggattacgctgaacagctatgtgctgcgtccgaagtctcgcggtaccgttcggctgcgttcggcagatccaagggtcaatccgatggtcgatcccaatttccttggagacccggccgaccttgagacgtctgcggaaggtgtgcggctgagctacgagatgttctcccagccttccttgcagaagcacatcaaggaaacatgcttctttagcggtaaacagccgacgatgcagatgtatcgggactatgcgcgggaacatggccggacctcctatcatccgacatgcacctgcaagatggggcgggatgacatgtccgtcgtcgatccgcgtctgaaggttcatggccttgagggcatcaggatctgtgacagctcggtcatgccgtcgctgctcggttccaacaccaatgccgcgacgatcatgatcagtgagcgggcagcggatttcattcaggggaacgcctga

SEQ ID NO: 2 - protein, 531 aa, Gluconobacter oxydans
MTSGFDYIVVGGGSAGCVLAARLSENPSVRVCLIEAGRRDTHPLIHMPVGFAKMTTGPHTWDLLTEPQKHANNRQIPYVQGRILGGGSSINAEVFTRGHPSDFDRWAAEGADGWSFRDVQKYFIRSEGNAVFSGTWHGTNGPLGVSNLADPNPTSRAFVQSCQEMGLPYNPDFNGASQEGAGIYQMTIRNNRRCSTAVGYLRPALGRKNLTVVTRALVLKIVFNGTRATGVQYIANGTLNTAEASQEIVVTAGAIGTPKLMMLSGVGPAAHLRENGIPVVQDLPGVGENLQDHFGVDIVAELKTDESFDKYRKLHWMLWAGLEYTMFRSGPVASNVVEGGAFWYSDPSSGVPDLQFHFLAGAGAEAGVTSVPKGASGITLNSYVLRPKSRGTVRLRSADPRVNPMVDPNFLGDPADLETSAEGVRLSYEMFSQPSLQKHIKETCFFSGKQPTMQMYRDYAREHGRTSYHPTCTCKMGRDDMSVVDPRLKVHGLEGIRICDSSVMPSLLGSNTNAATIMISERAADFIQGNA

SEQ ID NO: 3 - DNA, 20 bp, Artificial
  PCR-primer
atgacgagcggttttgatta

SEQ ID NO: 4 - DNA, 20 bp, Artificial
  PCR-primer
tcaggcgttcccctgaatga

SEQ ID NO: 5 - DNA, 33 bp, Artificial
  primer
ctcgagaagcttgatgactgcgtggccctgctg

SEQ ID NO: 6 - DNA, 59 bp, Artificial
  primer
ccctgaagaagaggatcaggccgtcgactctcactagtctccgtggtttcgggccggtc

SEQ ID NO: 7 - DNA, 59 bp, Artificial
  primer
gaccggcccgaaaccacggagactagtgagagtcgacggcctgatcctcttcttcaggg

SEQ ID NO: 8 - DNA, 31 bp, Artificial
  primer
ctcgatctagatgccgccaggtgcgtgggac

SEQ ID NO: 9 - DNA, 37 bp, Artificial
  primer
ctcgagaagctttggaacgttaagttcaatcttcacg

SEQ ID NO: 10 - DNA, 62 bp, Artificial
  primer
cgtggcataggtcttagatgacgtcgactctcgactagtgaccaagaactgttctggcaagg

SEQ ID NO: 11 - DNA, 62 bp, Artificial
  primer
ccttgccagaacagttcttggtcactagtcgagagtcgacgtcatctaagacctatgccacg

SEQ ID NO: 12 - DNA, 33 bp, Artificial
  primer
ctcgagtctagatgaatgctgctgatgagggag

SEQ ID NO: 13 - DNA, 34 bp, Artificial
  primer
ctcgagaagcttaaccttcttgtgacgggcgtgc

SEQ ID NO: 14 - DNA, 62 bp, Artificial
  primer
gtcctgtcagatcatttctgatcgtcgactctcactagtacggtgacttccggacaaagcac

SEQ ID NO: 15 - DNA, 62 bp, Artificial
  primer
gtgctttgtccggaagtcaccgtactagtgagagtcgacgatcagaaatgatctgacaggac

SEQ ID NO: 16 - DNA, 30 bp, Artificial
  primer
ctcgagtctagaccgccaattccggcagcg

SEQ ID NO: 17 - DNA, 34 bp, Artificial
  primer
ctcgagaagcttcaagatcgccatcccctatctg

SEQ ID NO: 18 - DNA, 59 bp, Artificial
  primer
gtccgtattcgatccgcatgggtcgactctcactagtgttcttactccgccatgccagc

SEQ ID NO: 19 - DNA, 59 bp, Artificial
  primer
gctggcatggcggagtaagaacactagtgagagtcgacccatgcggatcgaatacggac

SEQ ID NO: 20 - DNA, 33 bp, Artificial
  primer
ctcgagtctagatgtcctgttcagtctggggtg

SEQ ID NO: 21 - DNA, 33 bp, Artificial
  primer
ctcgagactagtaaacttggtctgacagttacc

SEQ ID NO: 22 - DNA, 48 bp, Artificial
  primer
gtcagggacgctgaggccactcgagccgctcatgagacaataaccctg

SEQ ID NO: 23 - DNA, 29 bp, Artificial
  primer
ctgactcgagtggcctcagcgtccctgac

SEQ ID NO: 24 - DNA, 38 bp, Artificial
  primer
ctcgaatcgataactaactcctgtgcgaactatggtgc

SEQ ID NO: 25 - DNA, 56 bp, Artificial
  primer
gcaccatagttcgcacaggagttagttatcgatgacgagcggttttgattacatcg

SEQ ID NO: 26 - DNA, 36 bp, Artificial
  primer
ctcgaggtcgactcaggcgttcccctgaatgaaatc

SEQ ID NO: 27 - DNA, 22 bp, Artificial
  primer
cgccggactgggcgatcgttgg

SEQ ID NO: 28 - DNA, 24 bp, Artificial
  primer
gccttttccagcgggggacgacca

SEQ ID NO: 29 - DNA, 19 bp, Artificial
  primer
tcgcaaccacccagaacac

SEQ ID NO: 30 - DNA, 21 bp, Artificial
  primer
tgtccacgaccagattagcca

SEQ ID NO: 31 - DNA, 20 bp, Artificial
  primer
aatcgtcccggctccggaaa

SEQ ID NO: 32 - DNA, 23 bp, Artificial
  primer
gcttgccgttgatcgcataggtg

SEQ ID NO: 33 - DNA, 18 bp, Artificial
  primer
agcttcgactggttctcc

SEQ ID NO: 34 - DNA, 20 bp, Artificial
  primer
agtacgaataggccgtgtag

SEQ ID NO: 35 - DNA, 24 bp, Artificial
  primer
gcatggaccagcttctcaagagcg

SEQ ID NO: 36 - DNA, 23 bp, Artificial
  primer
catgtgctggaacgtgaaattgc

SEQ ID NO: 37 - DNA, 21 bp, Artificial
  primer
caatgcgatagttcgtggacg

SEQ ID NO: 38 - DNA, 22 bp, Artificial
  primer
ggcattccggacatgaagaacg

SEQ ID NO: 39 - DNA, 23 bp, Artificial
  primer
ttgctcacccagaaacgctggtg

SEQ ID NO: 40 - DNA, 22 bp, Artificial
  primer
gtcatcgggtgttcctgatctc

SEQ ID NO: 41 - DNA, 22 bp, Artificial
  primer
gatttcctgcagcgcgtgcacc

SEQ ID NO: 42 - DNA, 24 bp, Artificial
  primer
acggcatgaattatggaacggttg

SEQ ID NO: 43 - DNA, 22 bp, Artificial
  primer
ggtcgatctgacagaggacggt

SEQ ID NO: 44 - DNA, 22 bp, Artificial
  primer
gtgtcgtatgtggttcccgagg

SEQ ID NO: 45 - DNA, 29 bp, Artificial
  primer
ctgactcgagttgaagtccgcgccgagcg

SEQ ID NO: 46 - DNA, 30 bp, Artificial
  primer
ctcgagtcgactttctccaaaaccccgctc

(19) United States
(12) Patent Application Publication
(10) Pub. No.: US 2012/0058563 A1
(43) Pub. Date: Mar. 8, 2012

(54) PRODUCTION OF 2-KETO-L-GULONIC ACID

(76) Inventors: Tatsuo Hoshino, Kanagawa (JP); Nigel John Mouncey, Indianapolis, IN (US); Akiko Shimizu, Kanagawa (JP); Masako Shinjoh, Kanagawa (JP)

(21) Appl. No.: 13/254,049

(22) PCT Filed: Mar. 4, 2010
(86) PCT No.: PCT/EP10/52770

§ 371 Date: Nov. 16, 2011

(30) Foreign Application Priority Data
Mar. 5, 2009 (EP) 09154412.2

(51) Int. Cl. C12N 15/74; C12N 1/00; C12N 1/21

(52) U.S. Cl. 435/471; 435/252.3; 435/243

(57) ABSTRACT

The present invention relates to the production of recombinant microorganisms, in particular of the genus Gluconobacter, for production of 2-keto-L-gulonic acid (2-KGA) and/or L-ascorbic acid (hereinafter also referred to as Vitamin C), wherein the microorganism has been modified to overexpress L-sorbose dehydrogenase (SDH). This overexpression has been achieved by introducing of one or more copies of a polynucleotide encoding SDH into the genome of the host microorganism resulting in enhanced yield, production, and/or efficiency of 2-KGA production and/or Vitamin C compared to a non-modified microorganism. Expression of said one or more extra-copies of sdh is dependent on the integration site. The invention also relates to genetically engineered microorganisms and their use for the production of 2-KGA and/or Vitamin C.

The present invention relates to the production of recombinant microorganisms, in particular of the genus Gluconobacter, for production of 2-keto-L-gulonic acid (2-KGA) and/or L-ascorbic acid (hereinafter also referred to as Vitamin C), wherein the microorganism has been modified to overexpress L-sorbose dehydrogenase (SDH). This overexpression has been achieved by introducing of one or more copies of a polynucleotide encoding SDH into the genome of the host microorganism resulting in enhanced yield, production, and/or efficiency of 2-KGA and/or Vitamin C production compared to a non-modified microorganism. Expression of said one or more extra-copies of sdh is dependent on the integration site. The invention also relates to genetically engineered microorganisms and their use for the production of 2-KGA and/or Vitamin C. Vitamin C is one of very important and indispensable nutrient factors for human beings. Vitamin C is also used in animal feed even though some farm animals can synthesize it in their own body.

For the past 70 years, Vitamin C has been produced industrially from D-glucose by the well-known Reichstein method. All steps in this process are chemical except for one (the conversion of D-sorbitol to L-sorbose), which is carried out by microbial conversion. Since its initial implementation for industrial production of Vitamin C, several chemical and technical modifications have been used to improve the efficiency of the Reichstein method. Recent developments of Vitamin C production are summarized in Ullmann's Encyclopedia of Industrial Chemistry, 5^{th} Edition, Vol. A27 (1996), pp. 547ff.

2-KGA is an important intermediate for the production of L-ascorbic acid. Microorganisms of the genus Acetobacter, Gluconobacter, or Pseudomonas are known for the production of 2-KGA from D-sorbitol. These microorganisms are capable of oxidizing D-sorbitol under aerobic condition producing 2-KGA.

2-KGA may be furthermore produced by a fermentation process starting from L-sorbose, by means of strains belonging e.g. to the Ketogulonicigenium or Gluconobacter genera, or by an alternative fermentation process starting from D-glucose, by means of recombinant strains belonging to the Gluconobacter or Pantoea genera.

The conversion of a substrate such as D-sorbitol into 2-KGA is a multistep-process involving several enzymes, such as e.g. dehydrogenases. The conversion of D-sorbitol to L-sorbose, for example, is catalyzed by D-sorbitol dehydrogenase (SLDH). L-Sorbose is further converted into L-sorbosone, catalyzed by L-sorbose dehydrogenase (SDH). Finally, L-sorbosone is converted to 2-KGA, which step is catalyzed by L-sorbosone dehydrogenase (SNDH). 2-KGA is further reduced into L-idonic acid, which is oxidized back to 2-KGA by L-idonate dehydrogenase (Hoshino et al. Agric. Biol. Chem. Vol. 54, No. 9, p. 2257-2263, 1990). Alternatively, L-sorbosone may be also directly converted to Vitamin C, which step is catalyzed by another type of SNDH.

Increase of 2-KGA and/or Vitamin C production from a given substrate can be done by e.g. increasing the activity of enzymes involved in the conversion process. An enzyme which has been selected as target for such experiments is SDH. Upon increasing the SDH-activity in a given microorganism, such as e.g. via introduction of multiple copies of sdh, one could increase the production of a target product such as e.g. 2-KGA or Vitamin C. However, the yield of target product can still be improved.

An object of the present invention is to improve the yields and/or productivity of 2-KGA and/or Vitamin C production.

Surprisingly, we now found that the increase of 2-KGA and/or Vitamin C production in a host cell carrying extra-copies of sdh is strongly dependent on the integration site in the host cell's genome. Suitable gene loci have been selected, wherein the disruption of the respective gene (for integration of sdh) does not have a negative effect on 2-KGA and/or Vitamin C production.

In particular, the object of the present invention is the generation of a microorganism, such as Gluconobacter, preferably Gluconobacter oxydans, that is diploid for the gene encoding SDH as a means to increase the oxidation of L-sorbose to L-sorbosone by the overexpression of sdh. The invention is directed to the introduction of one or more copies of the sdh gene into the genome of G. oxydans and expression thereof using different promoters. Suitable integration sites and promoters were shown to improve expression of SDH.

The polynucleotide encoding SDH useful for the present invention might be selected from known SDH-encoding genes, such as disclosed in e.g. EP 1846553 which has been isolated from Gluconobacter oxydans DSM 17078. Accordingly, the invention relates to a polynucleotide encoding an SDH protein integrated in the genome of a suitable host cell, wherein said polynucleotide being selected from the group consisting of:

(a) polynucleotides encoding a polypeptide comprising the amino acid sequence according to SEQ ID NO:2;
(b) polynucleotides comprising the nucleotide sequence according to SEQ ID NO:1;
(c) polynucleotides comprising a nucleotide sequence obtainable by nucleic acid amplification such as polymerase chain reaction, using genomic DNA from a microorganism as a template and a primer set according to SEQ ID NO:3 and SEQ ID NO:4;
(d) polynucleotides comprising a nucleotide sequence encoding a fragment or derivative of a polypeptide encoded by a polynucleotide of any of (a) to (c) wherein in said derivative one or more amino acid residues are conservatively substituted compared to said polypeptide, and said fragment or derivative has the activity of a sorbose dehydrogenase;
(e) polynucleotides the complementary strand of which hybridizes under stringent conditions to a polynucleotide as defined in any one of (a) to (d) and which encode a sorbose dehydrogenase; and
(f) polynucleotides which are at least 70%, such as 85, 90 or 95% identical to a polynucleotide as defined in any one of (a) to (d) and which encode a sorbose dehydrogenase; or the complementary strand of such a polynucleotide.

Polynucleotides according to SEQ ID NO:1, polynucleotides obtainable via PCR using primers according to SEQ ID NO:3 and 4, polynucleotides encoding a polypeptide according to SEQ ID NO:2, polynucleotides encoding fragments/derivatives of a polypeptide according to SEQ ID NO:2 containing conservatively substituted amino acid residues, polynucleotides hybridizing under stringent conditions to SEQ ID NO:1 and which encode SDH or polynucleotides which are at least 70, 85, 90 or 95% identical to said polynucleotides are in detail described in EP 1846553, see in particular page 17 line 6 to page 28 line 23 of said reference. The sdh shown in SEQ ID NO:1 has been isolated from G. oxydans DSM 17078.

Another SDH which may be used for the purpose of the present invention is the one isolated from G. oxydans T-100 disclosed in EP 753575 or as described by Saito et al. (Applied and Environmental Microbiology, Vol. 63, No. 2, p. 454-460, 1997).

Microorganisms which can be used for the present invention may be publicly available from different sources, e.g., Deutsche Sammlung von Mikroorganismen and Zellkulturen (DSMZ), Inhoffenstrasse 7B, D-38124 Braunschweig, Germany, American Type Culture Collection (ATCC), P.O. Box 1549, Manassas, Va. 20108 USA or Culture Collection Division, NITE Biological Resource Center, 2-5-8, Kazusakamatari, Kisarazu-shi, Chiba, 292-0818, Japan (formerly: Institute for Fermentation, Osaka (IFO), 17-85, Juso-honmachi 2-chome, Yodogawa-ku, Osaka 532-8686, Japan). Examples of preferred bacteria deposited with IFO are for instance Gluconobacter oxydans (formerly known as G. melanogenus) IFO 3293, Gluconobacter oxydans (formerly known as G. melanogenus) IFO 3292, Gluconobacter oxydans (formerly known as G. rubiginosus) IFO 3244, Gluconobacter frateurii (formerly known as G. industrius) IFO 3260, Gluconobacter cerinus IFO 3266, Gluconobacter oxydans IFO 3287, and Acetobacter aceti subsp. orleanus IFO 3259, which were all deposited on Apr. 5, 1954; Acetobacter aceti subsp. xylinum IFO 13693 deposited on Oct. 22, 1975, and Acetobacter aceti subsp. xylinum IFO 13773 deposited on Dec. 8, 1977. Strain Acetobacter sp. ATCC 15164, which is also an example of a preferred bacterium, was deposited with ATCC. Strain Gluconobacter oxydans (formerly known as G. melanogenus) N 44-1 as another example of a preferred bacterium is a derivative of the strain IFO 3293 and is described in Sugisawa et al., Agric. Biol. Chem. 54: 1201-1209, 1990. Furthermore, Gluconobacter oxydans (formerly known as G. albidus) IFO 3250, Gluconobacter oxydans (formerly known as G. albidus) IFO 3251, Gluconobacter oxydans (formerly known as G. albidus) IFO 3253, Gluconobacter oxydans (formerly known as G. suboxydans) IFO 3255, Gluconobacter oxydans (formerly known as G. cerinus) IFO 3263, Gluconobacter oxydans (formerly known as G. cerinus) IFO 3264, Gluconobacter oxydans (formerly known as G. cerinus) IFO 3265, Gluconobacter oxydans (formerly known as G. cerinus) IFO 3267, Gluconobacter oxydans (formerly known as G. cerinus) IFO 3268, Gluconobacter oxydans (formerly known as G. cerinus) IFO 3269, Gluconobacter oxydans (formerly known as G. melanogenus) IFO 3294, Gluconacetobacter liquefaciens (formerly known as Acetobacter liquefaciens) IFO 12257, and Gluconacetobacter liquefaciens (formerly known as Acetobacter liquefaciens) IFO 12258 can be used.

In particular, the present invention provides a process for the direct production of 2-KGA and/or Vitamin C comprising converting a substrate into 2-KGA and/or Vitamin C. This may for instance be done in a medium comprising a microorganism, which may be a resting or a growing microorganism. Suitable host cells as well as cultivation conditions including useful substrates have been described in EP 1846553, see in particular page 8 line 1 to page 17 line 5, wherein the conditions outlined for production of Vitamin C can be mutatis mutandis applied for 2-KGA production.

Preferred host cells are Gluconobacter or Acetobacter aceti, such as for instance G. oxydans, G. cerinus, G. frateurii, A. aceti subsp. xylinum or A. aceti subsp. orleanus, preferably G. oxydans DSM 17078.

In connection with the above process using a microorganism it is understood that the above-mentioned microorganisms also include synonyms or basonyms of such species having the same physiological properties, as defined by the International Code of Nomenclature of Prokaryotes. The nomenclature of the microorganisms as used herein is the one officially accepted (at the filing date of the priority application) by the International Committee on Systematics of Prokaryotes and the Bacteriology and Applied Microbiology Division of the International Union of Microbiological Societies, and published by its official publication vehicle International Journal of Systematic and Evolutionary Microbiology (IJSEM). A particular reference is made to Urbance et al., IJSEM (2001) vol 51:1059-1070, with a corrective notification on IJSEM (2001) vol 51:1231-1233, describing the taxonomic reclassification of G. oxydans DSM 4025 as Ketogulonicigenium vulgare.

As used herein, resting cells refer to cells of a microorganism which are for instance viable but not actively growing, or which are growing at low specific growth rates [μ], for instance, growth rates that are lower than 0.02 h⁻¹, preferably lower than 0.01 h⁻¹. Cells which show the above growth rates are said to be in a “resting cell mode”.

In connection with the above process using a microorganism, in the growth phase the specific growth rates are for instance at least 0.02 h⁻¹. For cells growing in batch, fed-batch or semi-continuous mode, the growth rate depends on for instance the composition of the growth medium, pH, temperature, and the like. In general, the growth rates may be for instance in a range from about 0.05 to about 0.2 h⁻¹, preferably from about 0.06 to about 0.151 h⁻¹, and most preferably from about 0.07 to about 0.13 h⁻¹.

As used herein, measurement in a “resting cell method” comprises (i) growing the cells by means of any method well know to the person skilled in the art, (ii) harvesting the cells from the growth broth, and (iii) incubating the harvested cells in a medium containing the substrate which is to be converted into the desired product, e.g. 2-KGA, under conditions where the cells do not grow any longer, i.e. there is no increase in the amount of biomass during this so-called conversion step.

In accordance with a further object of the present invention there is provided the use of a polynucleotide as defined above encoding a polypeptide having SDH activity or a microorganism which is genetically engineered using such polynucleotides in the production of 2-KGA and/or Vitamin C.

Modifications in order to have the host microorganism produce one or more copies of the SDH gene, i.e. overexpressing the gene, and/or protein may include the use of a strong promoter, or the mutation (e.g. insertion, deletion or point mutation) of (parts of) the SDH gene or its regulatory elements. It furthermore includes the insertion of multiple copies (or only a single copy) of the gene into a suitable microorganism, which may have SDH gene or may not have it. A gene is said to be “overexpressed” if the level of transcription of said gene is enhanced in comparison to the wild-type gene. This may be measured by for instance Northern blot analysis quantifying the amount of mRNA as an indication for gene expression. As used herein, a gene is overexpressed if the amount of generated mRNA is increased by at least 1%, 2%, 5% 10%, 25%, 50%, 75%, 100%, 200% or even more than 500%, compared to the amount of mRNA generated from a wild-type gene.

The present invention includes the step of altering a microorganism, wherein “altering” as used herein encompasses the process for “genetically altering” or “altering the composition of the cell culture media and/or methods used for culturing” in such a way that the yield and/or productivity of the fermentation product, in particular 2-KGA and/or Vitamin C, can be improved compared to the wild-type microorganism. As used herein, “improved yield of 2-KGA and/or Vitamin C” means an increase of at least 5%, 10%, 25%, 30%, 40%, 50%, 75%, 100%, 200% or even more than 500%, compared to a wild-type microorganism, i.e. a microorganism which is not genetically altered. When a microorganism having no functional sdh gene is used and an sdh gene is introduced by an integration into an integration site exemplified in this invention, the yield of 2-KGA and/or Vitamin C can be improved from no production to a significant level, which is shown below.

In connection with the above process using a microorganism, in one aspect, the process of the present invention leads to yields of 2-KGA which are at least about 1.8 g/l, preferably at least about 2.5 g/l, more preferably at least about 4.0 g/l, and most preferably at least about 5.7 g/l or more than 66 g/l. In one embodiment, the yield of 2-KGA produced by the process of the present invention is in the range of from about 1.8 to 600 g/l. The yield of 2-KGA refers to the concentration of 2-KGA in the harvest stream coming directly out of the production vessel, i.e. the cell-free supernatant comprising the 2-KGA.

The term “genetically engineered” or “genetically altered” means the scientific alteration of the structure of genetic material in a living organism, i.e. microorganism. It involves the production and use of recombinant DNA. More in particular it is used to delineate the genetically engineered or modified microorganism from the naturally occurring microorganism. Genetic engineering may be done by a number of techniques known in the art, such as e.g. gene replacement, gene amplification, gene disruption, transfection, transformation using plasmids, viruses, or other vectors. A genetically modified microorganism, e.g. genetically modified microorganism, is also often referred to as a recombinant microorganism.

The polypeptides and polynucleotides of the present invention are preferably provided in an isolated form, and preferably are purified to homogeneity.

The term “isolated” means that the material is removed from its original environment (e.g., the natural environment if it is naturally occurring). For example, a naturally-occurring polynucleotide or polypeptide present in a living microorganism is not isolated, but the same polynucleotide or polypeptide, separated from some or all of the coexisting materials in the natural system, is isolated. Such polynucleotides could be part of a vector and/or such polynucleotides or polypeptides could be part of a composition and still be isolated in that such vector or composition is not part of its natural environment.

An isolated polynucleotide or nucleic acid as used herein may be a DNA or RNA that is not immediately contiguous with both of the coding sequences with which it is immediately contiguous (one on the 5′-end and one on the 3′-end) in the naturally occurring genome of the organism from which it is derived. Thus, in one embodiment, a nucleic acid includes some or all of the 5′-non-coding (e.g., promoter) sequences that are immediately contiguous to the coding sequence. The term “isolated polynucleotide” therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant DNA that is part of a hybrid gene encoding an additional polypeptide that is substantially free of cellular material, viral material, or culture medium (when produced by recombinant DNA techniques), or chemical precursors or other chemicals (when chemically synthesized). Moreover, an “isolated nucleic acid fragment” is a nucleic acid fragment that is not naturally occurring as a fragment and would not be found in the natural state.

The terms “homology” or “percent identity” are used interchangeably herein. For the purpose of this invention, it is defined here that in order to determine the percent identity of two amino acid sequences or of two nucleic acid sequences, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

The skilled person will be aware of the fact that several different computer programs are available to determine the homology between two sequences. For instance, a comparison of sequences and determination of percent identity between two sequences may be accomplished using a mathematical algorithm. In a preferred embodiment, the percent identity between two amino acid sequences is determined using the Needleman and Wunsch (J. Mol. Biol. (48): 444-453 (1970)) algorithm which has been incorporated into the GAP program in the GCG software package (available at http://www.accelrys.com), using either a Blossom 62 matrix or a PAM250 matrix, and a gap weight of 16, 14, 12, 10, 8, 6 or 4 and a length weight of 1, 2, 3, 4, 5 or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the GCG software package (available at http://www.accelrys.com), using a NWSgapdna.CMP matrix and a gap weight of 40, 50, 60, 70 or 80 and a length weight of 1, 2, 3, 4, 5 or 6. In another embodiment, the percent identity between two amino acid or nucleotide sequences is determined using the algorithm of E. Meyers and W. Miller (CABIOS, 4: 11-17 (1989) which has been incorporated into the ALIGN program (version 2.0) (available at http://vega.igh.cnrs.fr/bin/align-guess.cgi) using a PAM120 weight residue table, a gap length penalty of 12 and a gap penalty of 4.

The nucleic acid and protein sequences of the present invention may further be used as a “query sequence” to perform a search against public databases to, for example, identify other family members or related sequences. Such searches may be performed using the BLASTN and BLASTX programs (version 2.0) of Altschul, et al. (1990) J. Mol. Biol. 215:403-10. BLAST nucleotide searches may be performed with the BLASTN program, score=100, word length=12 to obtain nucleotide sequences homologous to the nucleic acid molecules of the present invention. BLAST protein searches may be performed with the BLASTX program, score=50, word length=3 to obtain amino acid sequences homologous to the protein molecules of the present invention. To obtain gapped alignments for comparison purposes, Gapped BLAST may be utilized as described in Altschul et al., (1997) Nucleic Acids Res. 25 (17): 3389-3402. When utilizing BLAST and Gapped BLAST programs, the default parameters of the respective programs (e.g., BLASTX and BLASTN) may be used. See http://www.ncbi.nlm.nih.gov.

The sdh gene to be integrated into a suitable host cell may be operatively linked to an appropriate promoter, which may be either a constitutive or inducible promoter. The skilled person will know how to select suitable promoters. The expression constructs may contain sites for transcription initiation, termination, and, in the transcribed region, a ribosome binding site for translation. The coding portion of the mature transcripts expressed by the constructs may preferably include an initiation codon at the beginning and a termination codon appropriately positioned at the end of the polypeptide to be translated. Useful promoters and methods of cloning said promoters into a suitable vector are described in e.g. Saito et al. (see supra) or EP 453575. Preferably, the promoter can be selected from Psndh and PtufB. In addition, any promoters functional for a host selected can be used.

Vector DNA may be introduced into suitable host cells via conventional transformation or transfection techniques. As used herein, the terms “transformation”, “transconjugation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, transduction, infection, lipofection, cationic lipidmediated transfection or electroporation. Suitable methods for transforming or transfecting host cells may be found in Sambrook, et al. (supra), Davis et al., Basic Methods in Molecular Biology (1986) and other laboratory manuals.

In order to identify and select cells which have integrated the foreign DNA into their genome, a gene that encodes a selectable marker (e.g., resistance to antibiotics) is generally introduced into the host cells along with the gene of interest. Preferred selectable markers include those that confer resistance to drugs, such as kanamycin, tetracycline, ampicillin and streptomycin. A nucleic acid encoding a selectable marker is preferably introduced into a host cell on the same vector as the one encoding the protein according to the invention or can be introduced on a separate vector such as, for example, a suicide vector, which cannot replicate in the host cells. Cells stably transfected with the introduced nucleic acid can be identified by drug selection (e.g., cells that have incorporated the selectable marker gene will survive, while the other cells die). Alternatively, such a selective marker can be removed after integrating a foreign DNA into a genome, by a method using sacB system, whose technique is well known to the person skilled in the art.

The terms “production” or “productivity” are art-recognized and include the concentration of the fermentation product (for example, 2-KGA and/or Vitamin C) formed within a given time and a given fermentation volume (e.g., kg product per hour per liter). The term “efficiency of production” includes the time required for a particular level of production to be achieved (for example, how long it takes for the cell to attain a particular rate of output of a fermentation product). The term “yield” is art-recognized and includes the efficiency of the conversion of the carbon source into the product (i.e., 2-KGA and/or Vitamin C). This is generally written as, for example, kg product per kg carbon source. By “increasing the yield and/or production/productivity” of the compound it is meant that the quantity of recovered molecules, or of useful recovered molecules of that compound in a given amount of culture over a given amount of time is increased. The terms “biosynthesis” or a “biosynthetic pathway” are art-recognized and include the synthesis of a compound, preferably an organic compound, by a cell from intermediate compounds in what may be a multistep and highly regulated process. The language “metabolism” is art-recognized and includes the totality of the biochemical reactions that take place in an organism. The metabolism of a particular compound, then, (e.g., the metabolism of an amino acid such as glycine) comprises the overall biosynthetic, modification, and degradation pathways in the cell related to this compound. The language “transport” or “import” is art-recognized and includes the facilitated movement of one or more molecules across a cellular membrane through which the molecule would otherwise either be unable to pass or be passed inefficiently.

The present invention is concerned with the overexpression of one key enzyme involved in the fermentative production of 2-KGA and/or Vitamin C, namely overexpression of SDH. “Overexpression of SDH” includes introduction of one or more extra copies of sdh into a suitable microorganism defined herein, wherein said one or more copies are integrated into an endogenous plasmid or a gene locus on the chromosome of the host cell, whose integration does not inhibit a growth of the microorganism and expression of the sdh gene. An assay for measurement of SDH activity is described in e.g. Saito et al. (see supra) or in Sugisawa et al. (Agric. Biol. Chem. 55, p. 363-370, 1991).

In one embodiment, the one or more extra copies of sdh has/have been integrated into the gene locus of the L-sorbose reductase (SR) gene. SR catalyzes the conversion of L-sorbose into D-sorbitol and has been described by e.g. Shinjoh et al. (Journal of Bacteriology, Vol. 184, No. 3, p. 861-863, 2002) or in EP 1859031.

In a further embodiment, the one or more extra copies of sdh has/have been integrated into the gene locus of the 2-KGA reductase (KR) gene, described in e.g. Hoshino et al. (Agric. Biol. Chem. 54, p. 1211-1218, 1990), and Manning et al. (U.S. Pat. No. 5,082,785) who did not show actual example of introduction of SDH gene into the gene whose disruption with Tn5 resulted in no KR activity.

In another embodiment, the one or more extra copies of sdh has/have been integrated into the gene locus of the glucose dehydrogenase (GDH) gene. Genes encoding GDH have been described for instance in EP 1931785 or EP 1934337.

In one particular embodiment, the one or more extra copies of sdh has/have been integrated into the gene locus of the cytochrome bd oxidase (CydB) gene. An example of such an enzyme involved in the electron transport system and which could be used for the performance of the present invention is disclosed in WO 2006/084730.

Particularly, the one or more extra-copies of sdh are introduced into Gluconobacter, in particular Gluconobacter oxydans, preferably G. oxydans DSM 17078, wherein integration takes preferably place in at least one of the integration sites/gene loci mentioned above, i.e. sr, kr, gdh, and/or cydB gene locus. Methods for integration of foreign DNA into a microorganism such as, e.g. Gluconobacter oxydans, are known in the art and are exemplified in the Examples.

It had been surprisingly found out that integration of sdh into the kr, gdh, or cydB gene locus leads to the high production of 2-KGA together with the related products such as L-sorbosone, Vitamin C and L-idonic acid. Very low 2-KGA and the related products production was achieved with integration of sdh into the sr gene locus.

Integration constructs containing a sdh cassette could be furthermore combined with promoters, such as e.g. PtufB, instead of the natural promoter Psndh. However, it turned out that Psndh is the best promoter in connection with the herein described chromosomal integration of sdh when using a strain wherein the sdh gene has been disrupted (such as e.g. strain GO2026 derived from G. oxydans DSM 17078).

Measurement of 2-KGA or Vitamin C production may be performed by a method known in the art, in particular via Thin Layer Chromoatography (TLC) or High Performance Liquid Chromoatography (HPLC) analysis described herein. Any promoters naturally existing or the derivatives can be used for expressing an sdh gene in the suitable host microorganism.

Vitamin C as used herein may be any chemical form of L-ascorbic acid found in aqueous solutions, such as for instance undissociated, in its free acid form or dissociated as an anion. The solubilized salt form of L-ascorbic acid may be characterized as the anion in the presence of any kind of cations usually found in fermentation supernatants, such as for instance potassium, sodium, ammonium, or calcium. Also included may be isolated crystals of the free acid form of L-ascorbic acid. On the other hand, isolated crystals of a salt form of L-ascorbic acid are called by their corresponding salt name, i.e. sodium ascorbate, potassium ascorbate, calcium ascorbate and the like.

Vectors which may be useful for integration of sdh into the genome of the host cell without carrying the vector part are known in the art. One particular example of such a useful vector is pK18 (see http://www.ncbi.nlm.nih.gov/nuccore/207845). A vector useful in this invention can be a suicide plasmid that cannot replicate in a microorganism as a host, or a plasmid that cannot replicate under a certain condition such as higher temperature like e.g. 42° C. when the plasmid has a temperature-sensitive replication origin.

It will be appreciated by those skilled in the art that the design of the vector for integration of a desired polynucleotide fragment without the vector part can depend on such factors as the choice of the host cell to be transformed, the level of expression of protein desired, etc. The vectors for integration (hereinafter, integration vector) of the invention may be introduced into host cells to thereby facilitate a replacement of an integration site gene with a desired polynucleotide fragment having upstream and downstream flanking sequences of the integration site gene. This event can be done by either of two processes:

(1) Double crossing-over event occurring on the upstream and downstream flanking sequences at once.
(2) Integration of an integration vector having a desired polynucleotide fragment once in a chromosome or endogenous plasmid via a first single crossing-over event on either of the upstream and downstream flanking sequences, followed by a second single crossing-over event on the other flanking sequence to delete the vector part of the integration vector.

Both processes finally generate a recombinant microorganism having a desired polynucleotide fragment sequence instead of the integration gene sequence. The desired polynucleotide fragment sequence in this invention may be introduced into host cells to thereby proteins or peptides, encoded by nucleic acids as described herein, including, but not limited to, mutant proteins, fragments thereof, variants or functional equivalents thereof, and fusion proteins, encoded by a nucleic acid as described herein, e.g., SDH proteins, mutant forms of SDH proteins, fusion proteins and the like.

Advantageous embodiments of the invention become evident from the dependent claims. These and other aspects and embodiments of the present invention should be apparent to those skilled in the art from the teachings herein.

This invention is further illustrated by the following examples which should not be construed as limiting. The contents of all references, such as scientific literature, patent applications and patents, cited throughout this application are hereby incorporated by reference.

### FIGURE LEGENDS

FIG. 1. Construction of a vector having flanking regions FR1 and FR2 fragments. Primers used for PCR are indicated as p7-p10.

FIG. 2. Construction of Amp^{r}_Psndh and sdh cassettes. Primers used for PCR are indicated as p1-p6 (SEQ ID NOs:21-26).

FIG. 3. Construction of integration vector using pK18::FR1_FR2.

FIG. 4. Replacement of integration site for Amp^{r}_promoter_sdh cassette. The resulting plasmids are depicted on the right.

FIG. 5. PCR scheme to confirm different constructs as of Example 3.

FIG. 6. Polynucleotide sequence according to SEQ ID NO:1, i.e. sdh isolated from G. oxydans DSM 17078.

FIG. 7. Amino acids sequence according to SEQ ID NO:2, i.e. SDH isolated from G. oxydans DSM 17078.

FIG. 8. Polynucleotide sequence according to SEQ ID NO:3 and 4, i.e. primers for amplifying sdh according to SEQ ID NO:1.

### EXAMPLES

### Example 1

### Construction of Integration Vectors Carrying the L-Sorbose Dehydrogenase (SDH) Gene of Gluconobacter oxydans DSM 17078

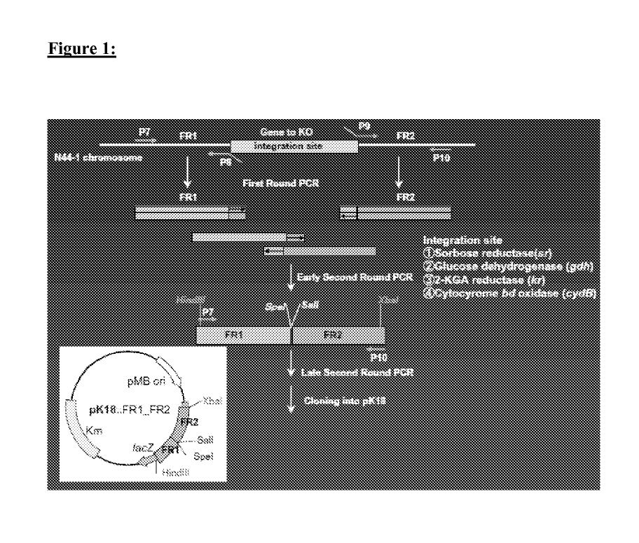

The following integration sites have been selected as target genes for integration of an extra-copy of the sdh gene in Gluconobacter oxydans DSM 17078: (a) sorbose reductase (sr) gene locus, (b) glucose dehydrogenase (gdh) gene locus, (c) 2-KGA reductase (kr) gene locus and (d) cytochrome bd oxidase (cydB) gene locus. Franking regions FR1 and FR2 of the target gene to be knocked out were cloned on pK18. In order to cut out the integration cassette later, HindIII and XbaI sites were designed. In the first round PCR (High Fidelity system Roche Diagnostics in a standard condition known by the skilled persons, such as “35 cycles of denaturation at 94° C. for 30 sec, annealing at 50° C. for 30 sec and extension at 72° C. for 1 min.”), primer pair p7/p8 were designed to prepare FR1 having partial sequence of the 5′-end of FR2, and primer pair p9/p10 to prepare FR2 having partial sequence of the 3′-end of FR1. Both products of the first round PCR were then ligated in the second round PCR using primer pair p7/p10 (High Fidelity system Roche Diagnostics in a standard condition known by the skilled persons, such as “94° C., 2 min, 10 cycles of [94° C., 30 sec, 63° C., 30 sec, 68° C., 6 min], followed by 20 cycles of [94° C., 30 sec, 63° C., 30 sec, 68° C., 6 min with an additional 20 sec per cycle] and a final extension at 68° C. for 10 min.”). Genomic DNA of G. oxydans DSM 17078 was used as template. At the junction of FR1 and FR2, SalI and SpeI restriction sites were designed to insert the Amp-promoter-sdh gene fragment to be prepared separately. Schematic diagram of the experiment is shown in FIG. 1. The following primer sequences were used for the different integration sites:

Primer sequences for FR1 and FR2 for knock-out of the sorbose reductase (SR) gene

| | |
|---|---|
| p7_sr (SEQ ID NO: 5): | ctcgagaagcttgatgactgcgtggccctgctg |
| p8_sr (SEQ ID NO: 6): | ccctgaagaagaggatcaggccgtcgactctcactagtctccgtggtttcgggccggtc |
| p9_sr (SEQ ID NO: 7): | gaccggcccgaaaccacggagactagtgagagtcgacggcctgatcctcttcttcaggg |
| p10_sr (SEQ ID NO: 8): | ctcgatctagatgccgccaggtgcgtgggac |

Primer sequences for FR1 and FR2 for knock-out of the 2-KGA reductase (KR) gene

| | |
|---|---|
| p7_kr (SEQ ID NO: 9): | ctcgagaagctttggaacgttaagttcaatcttcacg |
| p8_kr (SEQ ID NO: 10): | cgtggcataggtcttagatgacgtcgactctcgactagtgaccaagaactgttctggcaagg |
| p9_kr (SEQ ID NO: 11): | ccttgccagaacagttcttggtcactagtcgagagtcgacgtcatctaagacctatgccacg |
| p10_kr (SEQ ID NO: 12): | ctcgagtctagatgaatgctgctgatgagggag |

Primer sequences for FR1 and FR2 for knock-out of the glucose dehydrogenase (GDH) gene

| | |
|---|---|
| p7_gdh (SEQ ID NO: 13): | ctcgagaagcttaaccttcttgtgacgggcgtgc |
| p8_gdh (SEQ ID NO: 14): | gtcctgtcagatcatttctgatcgtcgactctcactagtacggtgacttccggacaaagcac |
| p9_gdh (SEQ ID NO: 15): | gtgctttgtccggaagtcaccgtactagtgagagtcgacgatcagaaatgatctgacaggac |
| p10_gdh (SEQ ID NO: 16): | ctcgagtctagaccgccaattccggcagcg |

Primer sequences for FR1 and FR2 for knock-out of the cytochrome bd oxidase (CydB) gene

| | |
|---|---|
| p7_cydB (SEQ ID NO: 17): | ctcgagaagcttcaagatcgccatcccctatctg |
| p8_cydB (SEQ ID NO: 18): | gtccgtattcgatccgcatgggtcgactctcactagtgttcttactccgccatgccagc |
| p9_cydB (SEQ ID NO: 19): | gctggcatggcggagtaagaacactagtgagagtcgacccatgcggatcgaatacggac |
| p10_cydB (SEQ ID NO: 20): | ctcgagtctagatgtcctgttcagtctggggtg |

Four kinds of integration vectors designated pK18::sr, pK18::kr, pK18::gdh and pK18::cydB were constructed. The vectors were introduced into G. oxydans DSM 17078 and replacement of the target gene by the FR1_FR2 fragment was confirmed via sequencing.

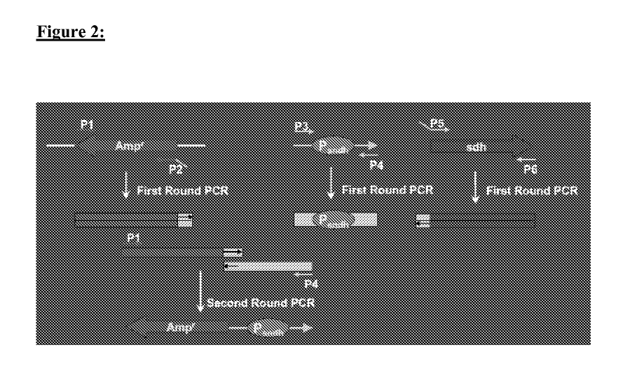

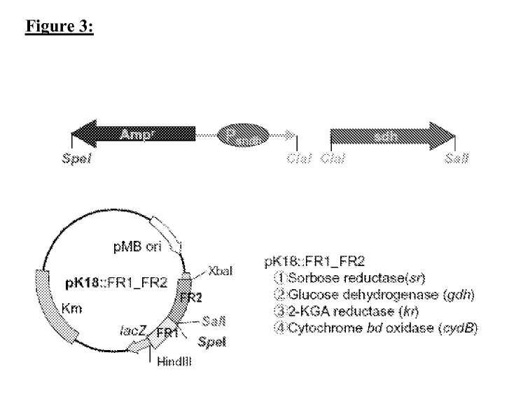

The integration cassette containing the extra-copy of the sdh gene and a strong promoter Psndh was constructed as follows: The amp^{r}_Psndh cassette having SpeI and ClaI restriction sites was prepared by PCR with the procedures shown in FIG. 2. Simultaneously, the sdh gene cassette having ClaI and SalI sites was prepared.

PCR-primers used were as follows (see FIG. 2):

p1 (SEQ ID NO: 21): ctcgagactagtaaacttggtctgacagttacc
p2 (SEQ ID NO: 22): gtcagggacgctgaggccactcgagccgctcatgagacaataaccctg
p3 (SEQ ID NO: 23): ctgactcgagtggcctcagcgtccctgac
p4 (SEQ ID NO: 24): ctcgaatcgataactaactcctgtgcgaactatggtgc
p5 (SEQ ID NO: 25): gcaccatagttcgcacaggagttagttatcgatgacgagcggttttgattacatcg
p6 (SEQ ID NO: 26): ctcgaggtcgactcaggcgttcccctgaatgaaatc

The amp^{r}_Psndh and the sdh cassette were cloned into the 4 integration vectors mentioned above and the intact sequence confirmed. The resulting vectors were named pK18::sr-amp^{r}_Psndh_sdh, pK18::kr-amp^{r}_Psndh_sdh, pK18::gdh-amp^{r}_Psndh_sdh, and pK18::cydB-amp^{r}_Psndh_sdh (see FIG. 3).

### Example 2

### Replacement of Psndh by a Constitutive Promoter

In order to further improve expression of sdh, the integration vectors as of Example 1 were combined with constitutive promoter PtufB (Saito et al. Applied and Environmental Microbiology, Vol. 63, No. 2, p. 454-460, 1997).

Promoter fragment was constructed via PCR, using primers prim3/prim4 together with the chromosomal DNA of G. oxydans DSM 17078 as the template:

prim3 (SEQ ID NO: 45):
ctgactcgagttgaagtccgcgccgagcg
prim4 (SEQ ID NO: 46):
ctcgagtcgactttctccaaaaccccgctc

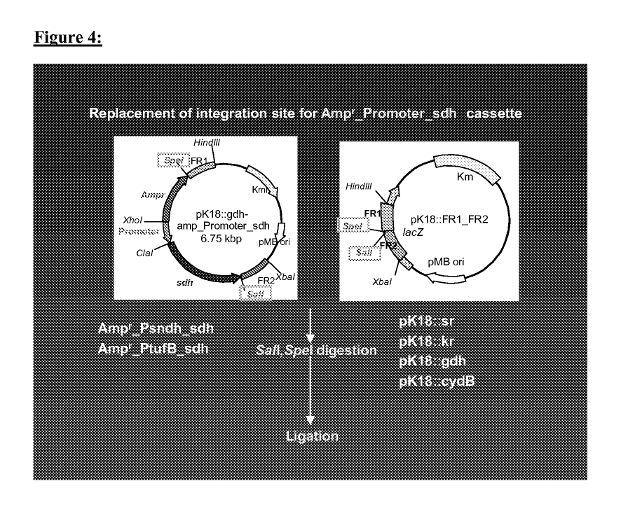

Since PtufB has ClaI site internally, AccI site was designed and ligated with ClaI site in case of the construction of the integration vector. PtufB was combined with the sdh gene cassette (see Example 1) and the obtained constructs were ligated with the respective integration vectors, leading to the following constructs: pK18::sr-amp^{r}_PtufB_sdh, pK18::kr-amp^{r}_PtufB_sdh, pK18::gdh-amp^{r}_PtufB_sdh, pK18::cydB-amp^{r}_PtufB_sdh. The method is schematically outlined in FIG. 4.

### Example 3

### Transformation of the Integration Cassettes into G. oxydans GO2026

Totally 8 different integration vectors have been obtained (see Ex. 1 and 2) which were used for transformation of competent cells of G. oxydans GO2026, a mutant based on G. oxydans DSM 17078 and wherein the natural sdh gene has been knocked out.

Single-cut vectors with no purification steps were directly used to transform G. oxydans GO2026, wherein pK18::sr-Amp^{r}_Psndh_sdh was linearized with EcoRI and pK18::kr (gdh or cydB)-Amp^{r}_Psndh_sdh was linearized with BglII.

The DNA fragments (100 or 400 ng) were added into 50 μl of the competent G. oxydans GO2026 cells. Electroporation pulse settings were 1.7 kV, 25 μF and 100Ω. After electroporation, the cells were suspended into 1 ml of MB medium, incubated at 29° C. for 3 hours with shaking (200 rpm), and 250 μl of the cell culture was spread on the MB agar plates containing 40 μg/ml each of Km and Amp (transformants containing the constitutive promoter: MB agar plates containing 50 μg/ml of Km and 40 μg/ml Amp) and those containing 40 μg/ml of Km and 20 μg/ml of Amp. After incubation for 3 days at 27° C., colonies were transferred into MB (liquid medium) containing 40 μg/ml of Km and 30 μg/ml of Amp and cultivated at 29° C. for 2 days with shaking (150 rpm).

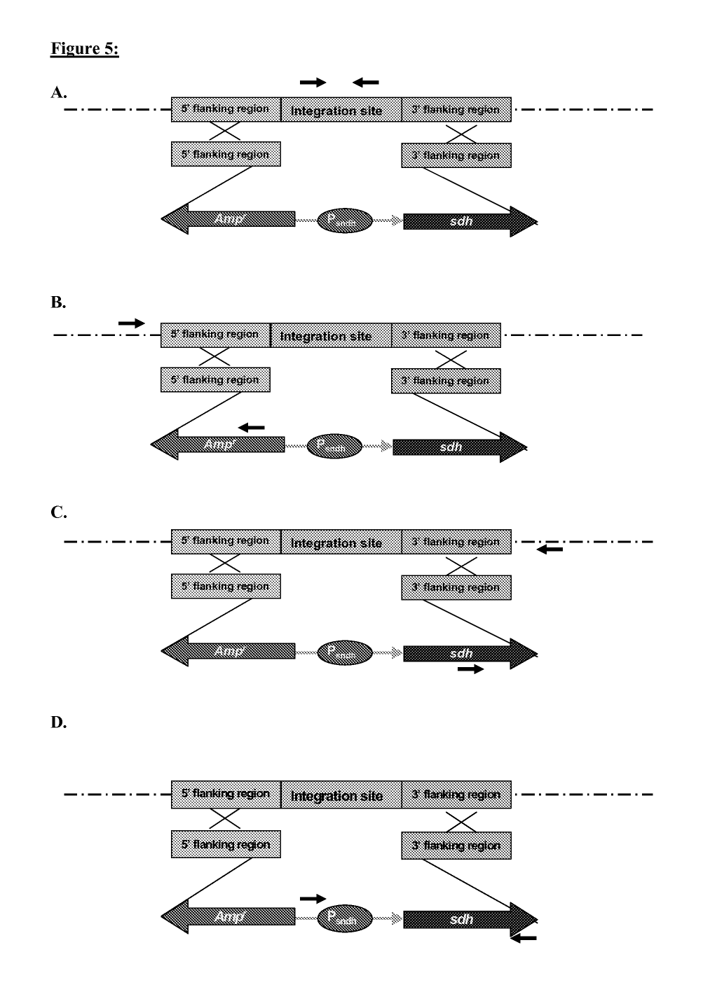

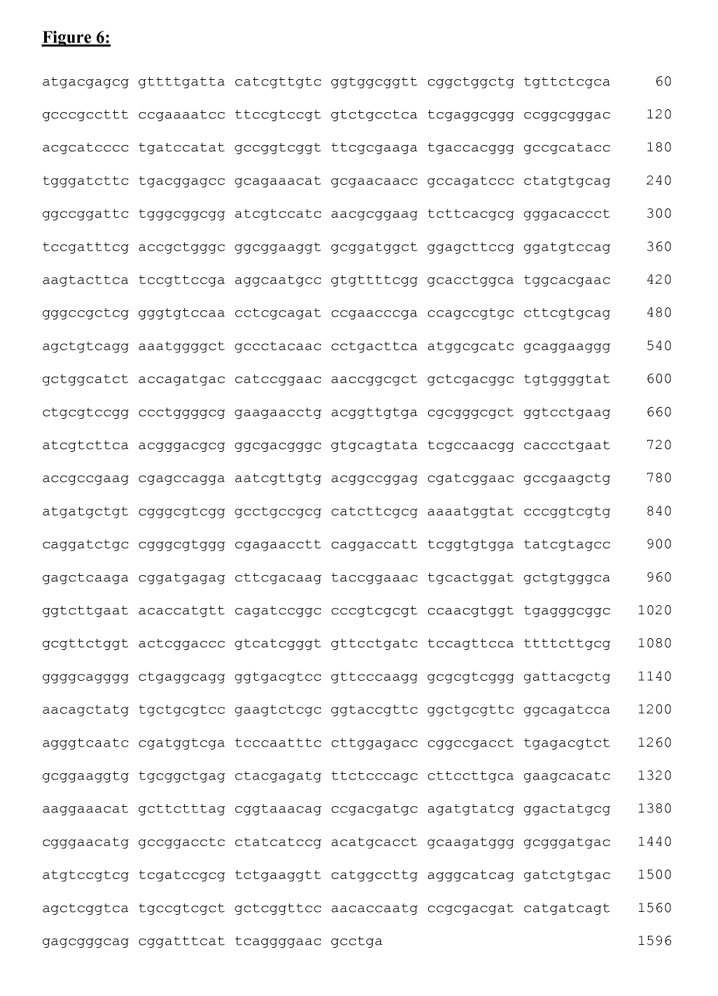

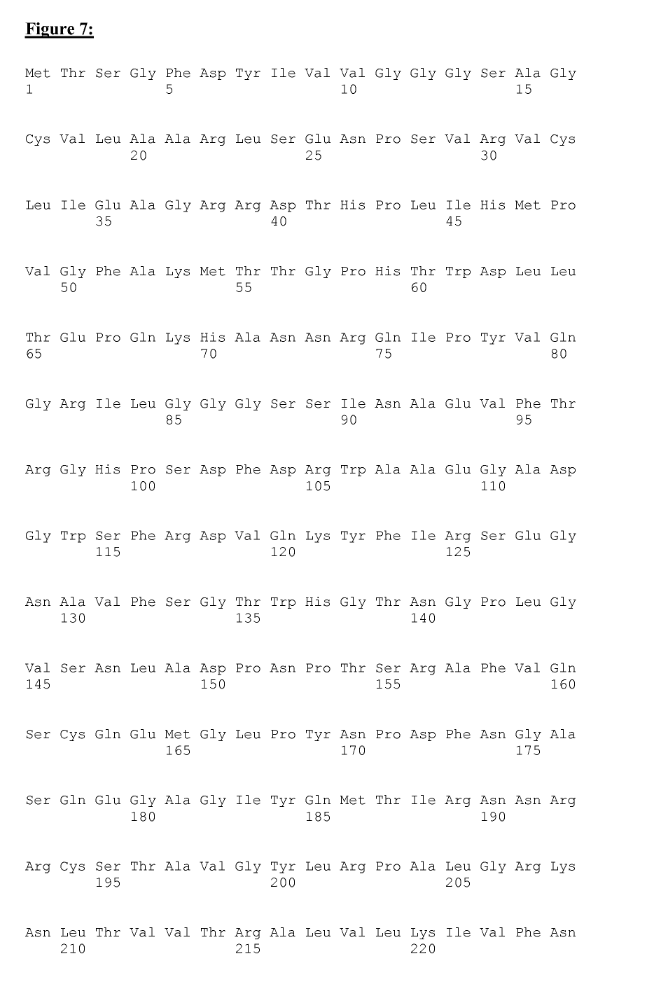

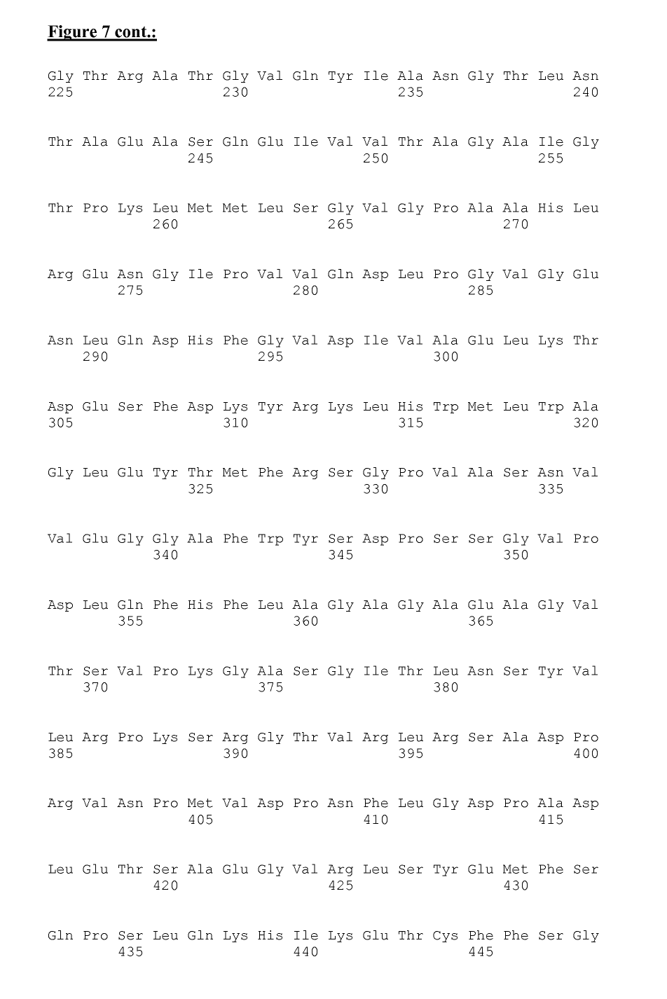

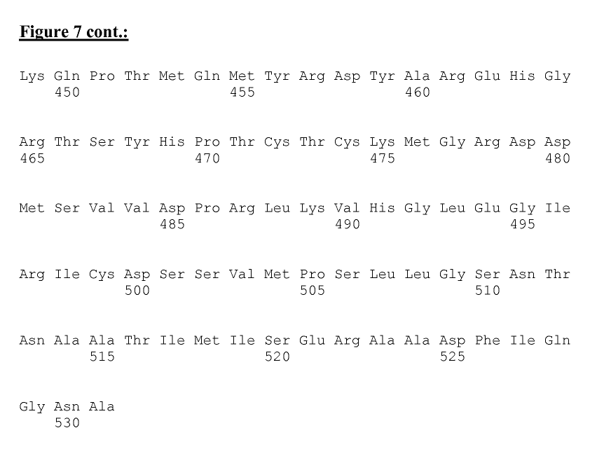

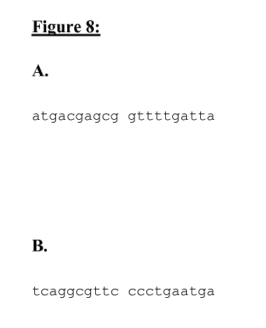

Confirmation of integration events was done by PCR amplification of 4 different loci around the integration part using chromosomal DNA of the transformants (see FIG. 5). Four different PCR procedures (A) to (D) were performed using the following primer pairs:

### (A) PCR to Confirm Absence of the Integration Site

| (SEQ ID NO: 27 and 28) | |
|---|---|
| sr_fwd | (cgccggactgggcgatcgttgg) |
| and | |
| sr_rev | (gccttttccagcgggggacgacca) for sr |
| (SEQ ID NO: 29 and 30) | |
| kr_fwd | (tcgcaaccacccagaacac) |
| and | |
| kr_rev | (tgtccacgaccagattagcca) for kr |
| (SEQ ID NO: 31 and 32) | |
| gdh_fwd | (aatcgtcccggctccggaaa) |
| and | |
| gdh_rev | (gcttgccgttgatcgcataggtg) for gdh |
| (SEQ ID NO: 33 and 34) | |
| cydB_fwd | (agcttcgactggttctcc) |
| and | |
| cydB_rev | (agtacgaataggccgtgtag) for cydB |

### (B) PCR to Confirm Recombination at the FR1 Site

sr_FR1_upstream
(gcatggaccagcttctcaagagcg; SEQ ID NO: 35) and amp_
fwd
(ttgctcacccagaaacgctggtg; SEQ ID NO: 39)
kr_FR1_upstream
(catgtgctggaacgtgaaattgc; SEQ ID NO: 36) and amp_
fwd
gdh_FR1_upstream
(caatgcgatagttcgtggacg; SEQ ID NO: 37) and amp_fwd
cydB_FR1_upstream
(ggcattccggacatgaagaacg; SEQ ID NO: 38) and amp_
fwd

### (C) PCR to Confirm Recombination at the FR2 Site

sdh_internal_fwd
(gtcatcgggtgttcctgatctc; SEQ ID NO: 40)
and
sr_ FR2_downstream
(gatttcctgcagcgcgtgcacc; 41)
sdh_internal_fwd and kr_FR2_downstream
(acggcatgaattatggaacggttg; SEQ ID NO: 42)
sdh_internal_fwd and gdh_FR2_downstream
(ggtcgatctgacagaggacggt; SEQ ID NO: 43)
sdh_internal_fwd and cydB_FR2_downstream
(gtgtcgtatgtggttcccgagg; SEQ ID NO: 44)

(D) PCR to Confirm Presence of the Promoter, e.g. Psndh: p3 and p6

### Example 4

### Production of 2-KGA in Resting Cell Reactions

2-KGA productivity of the integrants (see Ex. 3) were analyzed by the resting cell reaction system. 2-KGA and other metabolites were analyzed by TLC (Thin Layer Chromatography) and HPLC (High Performance Liquid Chromatography).

The integrants obtained in Example 3 were inoculated on MB agar plates containing 40 μg/ml each of Km and Amp, and incubated at 27° C. for 3 days. Colonies were further spread entirely on a petri dish with No. 3BD-7% sorbitol agar medium containing 40 μg/ml each of Km and Amp, and incubated at 27° C. for 3 days. The cell mass was then recovered, suspended in 500 μl of sterile water, diluted appropriately, and the OD at 600 nm was measured. Finally, the cell suspension having OD₆₀₀=20 was prepared and used for the resting cell reaction. The reaction mixture consisted of 250 μl of the cell suspension (OD₆₀₀=20), 50 μl of 20% sorbose solution, 125 μl of 4% CaCO₃+1.2% NaCl solution, 75 μl of sterile water. The reaction mixture was incubated at 30° C. with shaking (220 rpm) for 20 hours. The reaction mixture was centrifuged, and the supernatant was recovered for TLC analysis. Alternatively, the supernatant was mixed with an equal volume of 0.01 M H₂SO₄ and frozen until HPLC analysis.

For TLC analysis, 2 μl of either the sample or the standard (10 mg/ml) was applied on a TLC plate (Merck Silica gel 60 F254 5×20 cm) using as solvent n-propanol:H₂O:1% H₃PO₄:HCOOH=40:10:1:1. Detection of tetrabase, bluetetrazolium and naphtoresorcinol was as follows:

Tetrabase: spraying of 0.5% KIO₄, air-dry well, followed by spraying tetrabase-saturated solvent in 2N acetic acid:15% MnSO₄ in H₂O=1:1.

Bluetetrazolium: spraying of 0.5% bluetetrazolium in MeOH:6N NaOH=1:1 and heating at 100° C.

Naphtoresorcinol: spraying of 0.2% naphtoresorcinol in EtOH:conc. H₂SO₄=50:1 followed by heating at 100° C.

For all tested integrants, production 2-KGA and/or Vitamin C together with L-sorbosone and idonic acid was detected on TLC, meaning a reactivation of SDH activity by integration of the different constructs into mutant strain G. oxydans GO2026.

HPLC analysis was performed using an Agilent 1100 HPLC system (Agilent Technologies, Wilmington, USA) with a LiChrospher-100-RP18 (125×4.6 mm) column (Merck, Darmstadt, Germany) attached to an Aminex-HPX-78H (300×7.8 mm) column (Biorad, Reinach, Switzerland). The mobile phase is 0.004 M sulfuric acid with a flow rate of 0.6 ml/min. Two signals are recorded using an UV detector (wavelength 254 nm) in combination with a refractive index detector. In addition, the identification of the L-ascorbic acid is done using an amino-column (YMC-Pack Polyamine-II, YMC, Inc., Kyoto, Japan) with UV detection at 254 nm. The mobile phase is 50 mM NH₄H₂PO₄ and acetonitrile (40:60).

HPLC assay confirmed that integration of the sdh cassette including Psndh as a promoter in all the four integration sites, sr, kr, gdh, and cydB genes, resulted in a production of 2-KGA and/or Vitamin C together with L-sorbosone and idonic acid. The integrants produced SDH-related products (L-sorbosone, 2-KGA, Vitamin C, and L-idonic acid) in the range of 30 to 80% of those produced by G. oxydans DSM 17078, whereas the host strain G. oxydans GO2026 produced none of them. Integration of the sdh cassette using kr, gdh, and cydB genes were especially suitable for production of 2-KGA and/of Vitamin C, whereas the one using the sr gene was less suitable. Integration of the sdh cassette including PtufB as a promoter also resulted in production of the SDH-related products when it was integrated in kr, gdh, and cydB gene in the range of 1-5% of those produced by G. oxydans DSM 17078.

1.-10. (canceled)

11. A recombinant microorganism comprising an integrated polynucleotide fragment containing a polynucleotide encoding a protein having L-sorbose dehydrogenase (SDH) activity, said polynucleotide being selected from the group consisting of:
(a) polynucleotides encoding a polypeptide comprising the amino acid sequence according to SEQ ID NO:2;
(b) polynucleotides comprising the nucleotide sequence according to SEQ ID NO:1;
(c) polynucleotides comprising a nucleotide sequence obtainable by nucleic acid amplification such as polymerase chain reaction, using genomic DNA from a microorganism as a template and a primer set according to SEQ ID NO:3 and SEQ ID NO:4;
(d) polynucleotides comprising a nucleotide sequence encoding a fragment or derivative of a polypeptide encoded by a polynucleotide of any of (a) to (c) wherein in said derivative one or more amino acid residues are conservatively substituted compared to said polypeptide, and said fragment or derivative has the activity of a SDH polypeptide;
(e) polynucleotides the complementary strand of which hybridizes under stringent conditions to a polynucleotide as defined in any one of (a) to (d) and which encode a SDH polypeptide;
(f) polynucleotides which are at least 70%, such as 85, 90 or 95% homologous to a polynucleotide as defined in any one of (a) to (d) and which encode a SDH polypeptide;
or the complementary strand of such a polynucleotide and wherein said polynucleotide being integrated into the genome of the recombinant microorganism and wherein the integration site is selected from the group consisting of L-sorbose reductase gene locus, 2-keto-L-gulonic acid reductase gene locus, glucose dehydrogenase gene locus and cytochrome bd oxidase gene locus.

12. The microorganism according to claim 11, wherein the sdh gene is further combined with an exogenous promoter sequence.

13. The microorganism according to claim 11, wherein the integration of the sdh gene does not inhibit the growth or microorganism and expression of the sdh gene.

14. The microorganism according to claim 11, wherein the microorganism is selected from Gluconobacter, Gluconoacetobacter and Acetobacter.

15. The microorganism according to claim 14 which is Gluconobacter oxydans, in particular Gluconobacter oxydans DSM 17078.

16. A process for generation of a recombinant microorganism comprising the steps of:
(a) generation of an integration vector comprising one or more copies of sdh gene cassette together with upstream and downstream flanking polynucleotide sequences of an integration site
(b) generation of a knock out of a putative integration site gene via replacement of said integration site gene by introduction of the integration vector of (a), wherein the integration site gene is selected from the group consisting of L-sorbose reductase gene, 2-keto-L-gulonic acid reductase gene, glucose dehydrogenase gene, and cytochrome bd oxidase gene.

17. A process according to claim 16, wherein after step (a) a promoter is cloned in front of the L-sorbose dehydrogenase gene prior to introduction of the L-sorbose dehydrogenase gene into the integration site.

18. A process according to claim 16, wherein after step (a) a marker gene is cloned upstream or downstream of the L-sorbose dehydrogenase gene prior to introduction of the L-sorbose dehydrogenase gene into the integration site.

19. A process for the production of 2-keto-L-gulonic acid and/or Vitamin C using a microorganism according to claim 11.
